(19) United States
(12) Patent Application Publication
(10) Pub. No.: US 2021/0137328 A1
(43) Pub. Date: May 13, 2021

(54) AUTOMATIC CLEANING APPARATUS

(71) Applicant: NKFG Corporation, Taipei City (TW)

(72) Inventors: Te-Li TIEN, New Taipei City (TW); Shao-Wei CHIU, Hsinchu City (TW); Yu-De LIN, New Taipei City (TW); Hsiang-En CHIU, New Taipei City (TW); Han-Chien CHENG, Taipei City (TW); Chih-Chang LIN, Kaohsiung City (TW)

(73) Assignee: NKFG Corporation, Taipei City (TW)

(21) Appl. No.: 16/880,642

(22) Filed: May 21, 2020

(30) Foreign Application Priority Data
Nov. 7, 2019 (TW) 108140383

(51) Int. Cl. A47L 7/00; A47L 9/28; A61L 2/24; A61L 2/10

(52) U.S. Cl. CPC A47L 7/0061; A61L 2202/25; A47L 9/2894; A47L 9/2852; A47L 9/2889; A47L 9/2847; A61L 2/24; A61L 2/10; A47L 2201/04; A47L 2201/06; A61L 2202/11; A61L 2202/14; A61L 2202/16; A61L 2202/17; A47L 9/2826

(57) ABSTRACT

An automatic cleaning apparatus is configured to clean a floor and emit ultraviolet light for sterilizing the floor in a cleaning mode. Upon detecting that a wall is nearby, the automatic cleaning apparatus emits ultraviolet light toward a corner formed by the floor and the wall for sterilization.

### CROSS-REFERENCE TO RELATED APPLICATION

This application claims priority of Taiwanese Invention Patent Application No. 108140383, filed on Nov. 7, 2019.

### FIELD

The disclosure relates to an automatic cleaning apparatus, and more particularly to an automatic cleaning apparatus that has a function of sterilization.

### BACKGROUND

Recently, automatic cleaning apparatuses, such as robot vacuums, robot mops, etc., have become popular since people wish to reduce time spent on cleaning. One goal in the relevant industry is to integrate more functions into such automatic cleaning apparatuses.

### SUMMARY

Therefore, an object of the disclosure is to provide an automatic cleaning apparatus that has a function of sterilization.

According to the disclosure, the automatic cleaning apparatus is adapted for cleaning a room, and includes a housing, a driving unit, a cleaning unit, a sensing unit, an ultraviolet (UV) light emitting diode (LED) unit, and a control unit. The housing has a bottom surface to be proximate to a floor of the room. The driving unit is disposed on the housing and is partly exposed from the bottom surface of the housing. The driving unit is operable to drive movement of the housing. The cleaning unit is disposed on the housing and is partly exposed from the bottom surface of the housing. The cleaning unit is configured to clean the floor. The sensing unit is disposed on the housing, and is configured to generate a trigger signal upon detecting that the housing is close to a wall of the room. The UV LED unit is disposed on the housing and is partly exposed from the bottom surface of the housing. The UV LED unit is operable to emit UV light for sterilization. The control unit is disposed within the housing and is electrically coupled to the driving unit, the cleaning unit, the sensing unit and the UV LED unit. The control unit is configured to, when operating in a cleaning mode: i) control the driving unit to drive movement of the housing based on a predetermined procedure; ii) control the cleaning unit to clean the floor; iii) control the UV LED unit to emit UV light toward the floor when the control unit does not receive the trigger signal from the sensing unit; and iv) control the UV LED unit to emit UV light toward a corner formed between the floor and the wall upon receipt of the trigger signal from the sensing unit.

### BRIEF DESCRIPTION OF THE DRAWINGS

Other features and advantages of the disclosure will become apparent in the following detailed description of the embodiment (s) with reference to the accompanying drawings, of which:

FIG. 1 is a block diagram illustrating an embodiment of an automatic cleaning apparatus according to the disclosure;

FIG. 2 is a bottom view of the embodiment;

FIG. 3 is a schematic diagram illustrating operation of the embodiment;

FIG. 4 is a sectional side view of the embodiment with sectional lines omitted; and

FIG. 5 is a fragmentary, partly see-through diagram of the embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Referring to FIGS. 1 and 2, an embodiment of an automatic cleaning apparatus is adapted for cleaning a room, and includes a housing 1, a control unit 2, a driving unit 3, a cleaning unit 4, a sensing unit 5, and an ultraviolet (UV) light emitting diode (LED) unit 6. In this embodiment, the automatic cleaning apparatus is exemplified as a robot vacuum, but this disclosure is not limited in this respect. For example, the automatic cleaning apparatus may be a robot mop, or any other kinds of self-propelled electronic cleaning devices in other embodiments.

Further referring to FIG. 3, the housing 1 has a bottom surface 11 to be proximate to a floor 91 in the room. The driving unit 3 is disposed on the housing 1, is partly exposed from the bottom surface 11, and is operable to drive movement of the housing 1. The driving unit 3 may include, but not limited to, a motor (not shown), multiple gears (not shown) that act with operation of the motor, an electronic circuit (not shown) to control the operation of the motor, and multiple wheels 31 linked to and driven by the gears, so the movement and the propelling direction of the housing 1 can be controlled.

Further referring to FIGS. 4 and 5, the cleaning unit 4 is disposed on the housing 1, is partly exposed from the bottom surface 11, and is configured to clean the floor 91. For example, the cleaning unit 4 may include, but not limited to, a motor (not shown), multiple gears (not shown) that act with operation of the motor, a vacuum device (not shown), an electronic circuit (not shown) to control the operation of the motor and the vacuum, a brush 41 linked to and driven by the gears, and a dust bin (not shown). It is noted that the brushes 41 are omitted from FIG. 2 for the sake of clarity. When the brushes 41 are driven to rotate, dusts and debris on the floor may be kicked up for suction by the vacuum. The cleaning unit 4 may include other kinds of cleaning components in other embodiments, such as a mopping component, and this disclosure is not limited in this respect.

The sensing unit 5 is disposed on the housing 1, and is configured to generate a trigger signal upon detecting that the housing 1 is close to a wall 92 (or some other obstacle) in the room. In this embodiment, the sensing unit 5 includes multiple optical distance sensors 51 and multiple light beam transmitters (not shown) that respectively correspond to the optical distance sensors 51. Each optical distance sensor 51 is configured to generate the trigger signal upon detecting that a distance between the housing 91 and the wall 92 is smaller than a predetermined distance in response to receipt of a signal transmitted by the corresponding light beam transmitter and reflected by the wall 92. For example, the predetermined distance may be between 1 centimeter and 5 centimeters, but this disclosure is not limited thereto. In one embodiment, the optical distance sensors 51 may be infrared distance sensors, and the light beam transmitters may be infrared transmitters that emit infrared light. In one embodiment, the optical distance sensors 51 may be laser distance sensors, and the light beam transmitters may be laser transmitters that emit laser beam. This disclosure is not limited to the type and the number of distance sensors, which may be decided depending on the developer's requirements. For example, ultrasonic sensors may be used in one embodiment, and only one distance sensor may be used in another embodiment.

The UV LED unit 6 is disposed on the housing 1, is partly exposed from the bottom surface 11, and is configured to emit UV light for sterilization purposes. In this embodiment, the UV LED unit 6 is exemplified to include multiple first UV light emitting diodes 61 (first group of UV light emitting diodes), multiple second UV light emitting diodes 62 (second group of UV light emitting diodes), and a corresponding driving circuit (not shown). The first UV light emitting diodes 61 are disposed on an inner portion of the bottom surface 11 (more specifically, near a central portion of the bottom surface 11), are away from an edge of the bottom surface 11, and are to sterilize the floor 91. The second UV light emitting diodes 62 are disposed proximate to the edge of the bottom surface 11 of the housing 1, and are to sterilize the floor 91 and the wall 92. It is noted that the UV LED unit 6 does not necessarily have to include the first UV light emitting diodes 61 and the second UV light emitting diodes 62 at the same time. In addition, the number of the first UV light emitting diodes 61 and the number of the second UV light emitting diodes 62 are not limited in this disclosure. For example, the UV LED unit 6 may include only one first UV light emitting diode 61 and/or only one second UV light emitting diode 62 in some embodiments.

The control unit 2 may be, for example, a microcontroller unit (MCU), and is disposed within the housing 1. The control unit 2 is electrically coupled to the driving unit 3, the cleaning unit 4, the sensing unit 5 and the UV LED unit 6, and is configured to be operable in a cleaning mode or a sterilization mode. In the cleaning mode, the control unit 2 controls the driving unit 3 to drive movement of the housing 1 based on a predetermined procedure (e.g., a conventional path planning algorithm); controls the cleaning unit 4 to clean the floor 91; controls the first and second UV light emitting diodes 61, 62 to emit UV light toward the floor 91 when the control unit 2 does not receive the trigger signal from the sensing unit 5; and controls the second UV light emitting diodes 62 to emit UV light toward a corner formed between the floor 91 and the wall 92 upon receipt of the trigger signal from the sensing unit 5.

When the control unit 2 receives the trigger signal in the cleaning mode, the control unit 2 controls the driving unit 3 to temporarily stop movement of the housing 1 for a predetermined period of time to allow time for the UV LED unit 6 to perform sterilization, followed by resuming the movement of the housing 1. The predetermined period of time may be set to be within a range of between 1 second and 30 seconds in order to effectively remove specific viruses and/or germs, and the setting thereof may be pre-stored in the control unit 2 or be manually defined by users.

As exemplified in FIG. 3, the first UV light emitting diodes 61 may be configured to emit UV light toward a first direction (L1) substantially or nearly perpendicular to the bottom surface 11. In a normal condition, the bottom surface 11 may be substantially parallel to the floor 91, so the UV light emitted by the first UV light emitting diodes 61 would be directly projected to the floor 91, achieving a better effect of sterilization. Upon receipt of the trigger signal, the control unit 2 controls the second UV light emitting diodes 62 to, during the predetermined period of time, emit UV light toward a second direction (L2) that cooperates with the first direction (L1) to forma fixed predetermined angle of between 0 degrees and 90 degrees, thereby illuminating the corner formed by the floor 91 and the wall 92. In some cases, the UV LED unit 6 may further include a driving motor and a driving circuit that are coupled to the control unit 2 and the second UV light emitting diodes 62, so the control unit 2 may, during the predetermined period of time, control the second UV light emitting diodes 62 to swing in such a way that a direction toward which the UV light emitted by the second UV light emitting diodes 62 varies within a predetermined range (e.g., an angle formed by such direction and the first direction varies between 0 degrees and 90 degrees, but this disclosure is not limited to such).

In some embodiments, the UV LED unit 6 may further include a driving motor and a driving circuit that are coupled to the control unit 2 and the first UV light emitting diodes 61, so the control unit 2 may control the first UV light emitting diodes 61 to swing in such a way that a direction toward which the UV light emitted by the first UV light emitting diodes 61 varies within a predetermined range (e.g., an angle formed by such direction and the first direction varies between 60 degrees and −60 degrees, but this disclosure is not limited to such).

In some embodiments, the control unit 2 controls the UV LED unit to emit UV light at a fixed predetermined intensity. In some embodiments, the control unit 2 controls the UV LED unit 6 to emit UV light at a first predetermined intensity when the control unit 2 does not receive the trigger signal from the sensing unit 5, and controls the UV LED unit 6 to emit UV light at a second predetermined intensity greater than the first predetermined intensity upon receipt of the trigger signal from the sensing unit 5 (e.g., for the predetermined period of time), so as to achieve stronger sterilization for the corner.

In the sterilization mode, the control unit 2 controls the driving unit 3 to drive movement of the housing 1 based on the predetermined procedure; controls the cleaning unit 4 to not operate; controls the first and second UV light emitting diodes 61, 62 to emit UV light toward the floor 91 when the control unit 2 does not receive the trigger signal from the sensing unit 5; and controls the second UV light emitting diodes 62 to emit UV light toward the corner formed between the floor 91 and the wall 92 upon receipt of the trigger signal from the sensing unit 5.

Referring to FIGS. 2, 4 and 5, in this embodiment, the housing 1 is formed with a ventilation channel 12 proximate to the UV LED unit 6, so airflow induced in the ventilation channel 12 during the movement of the housing 1 may dissipate heat generated by the UV LED unit 6. In FIGS. 2, 4 and 5, arrows 93 are used to indicate directions of the airflow in the ventilation channel 12. It is noted that the housing 1 may have one or multiple ventilation channels 12 according to this disclosure.

To sum up, the embodiment of the automatic cleaning apparatus according to this disclosure may perform cleaning and sterilization at the same time. In the cleaning mode, the control unit 2 controls the cleaning unit 4 and the UV LED unit 6 to clean and sterilize the floor 91. Upon receipt of the trigger signal, the control unit 2 controls the UV LED unit 6 to sterilize the corner formed by the floor 91 and the wall 92. When no cleaning but only sterilization is required, the control unit 2 can be set to operate in the sterilization mode, and perform only the sterilization. Furthermore, the ventilation channel 12 can effectively dissipate heat generated by the UV LED unit 6, ensuring normal operation of the automatic cleaning apparatus.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An automatic cleaning apparatus adapted for cleaning a room, comprising:
a housing having a bottom surface to be proximate to a floor of the room;
a driving unit disposed on said housing and partly exposed from said bottom surface of said housing, said driving unit being operable to drive movement of said housing;
a cleaning unit disposed on said housing and partly exposed from said bottom surface of said housing, said cleaning unit being configured to clean the floor;
a sensing unit disposed on said housing, and configured to generate a trigger signal upon detecting that said housing is close to a wall of the room;
an ultraviolet (UV) light emitting diode (LED) unit disposed on said housing and partly exposed from said bottom surface of said housing, said UV LED unit being operable to emit UV light for sterilization; and
a control unit disposed within said housing and electrically coupled to said driving unit, said cleaning unit, said sensing unit and said UV LED unit, said control unit being configured to, when operating in a cleaning mode: i) control said driving unit to drive movement of said housing based on a predetermined procedure; ii) control said cleaning unit to clean the floor; iii) control said UV LED unit to emit UV light toward the floor when said control unit does not receive the trigger signal from said sensing unit; and iv) control said UV LED unit to emit UV light toward a corner formed between the floor and the wall upon receipt of the trigger signal from said sensing unit.

2. The automatic cleaning apparatus of claim 1, wherein said sensing unit includes an infrared distance sensor configured to generate the trigger signal upon detecting that a distance between the wall and said housing is smaller than a predetermined distance.

3. The automatic cleaning apparatus of claim 1, wherein said sensing unit includes a laser distance sensor configured to generate the trigger signal upon detecting that a distance between the wall and said housing is smaller than a predetermined distance.

4. The automatic cleaning apparatus of claim 1, wherein said control unit is configured to, upon receipt of the trigger signal, control said driving unit to stop movement of said housing for a predetermined period of time, followed by resuming the movement of said housing.

5. The automatic cleaning apparatus of claim 1, wherein said UV LED unit includes a plurality of UV light emitting diodes, which are divided into a first group of said UV light emitting diodes disposed on an inner portion of said bottom surface of said housing, and configured to emit UV light toward a first direction substantially perpendicular to the bottom surface, and a second group of said UV light emitting diodes disposed proximate to an edge of said bottom surface of said housing, and configured to emit UV light toward a second direction that cooperates with the first direction to form an angle of between 0 degrees and 90 degrees.

6. The automatic cleaning apparatus of claim 1, wherein said UV LED unit includes a plurality of UV light emitting diodes, which are divided into a first group of said UV light emitting diodes disposed on an inner portion of said bottom surface of said housing, and configured to emit UV light toward a first direction substantially perpendicular to the bottom surface, and a second group of said UV light emitting diodes disposed proximate to an edge of said bottom surface of said housing, and configured to emit UV light;
wherein said control unit is configured to, upon receipt of the trigger signal, control said driving unit to stop movement of said housing for a predetermined period of time, during which said control unit controls said second group of said UV light emitting diodes to emit UV light, and to swing in such a way that a direction toward which the UV light emitted thereby varies within a predetermined range.

7. The automatic cleaning apparatus of claim 1, wherein said UV LED unit includes at least one UV light emitting diode that is disposed on an inner portion of said bottom surface of said housing, and that is configured to emit UV light and to swing in such a way that a direction toward which the UV light emitted thereby varies within a predetermined range.

8. The automatic cleaning apparatus of claim 1, wherein said control unit is further configured to, when operating in a sterilization mode: i) control said driving unit to drive movement of said housing based on the predetermined procedure; ii) control said cleaning unit to not operate; iii) control said UV LED unit to emit UV light toward the floor when said control unit does not receive the trigger signal from said sensing unit; and iv) control said UV LED unit to emit UV light toward the corner formed between the floor and the wall upon receipt of the trigger signal from said sensing unit.

9. The automatic cleaning apparatus of claim 1, wherein said housing is formed with a ventilation channel proximate to said UV LED unit, so as to induce airflow in said ventilation channel during the movement of said housing in order to dissipate heat generated by said UV LED unit.

10. The automatic cleaning apparatus of claim 1, wherein said control unit is configured to control said UV LED unit to emit UV light at a first predetermined intensity when said control unit does not receive the trigger signal from said sensing unit, and to control said UV LED unit to emit UV light at a second predetermined intensity greater than the first predetermined intensity upon receipt of the trigger signal from said sensing unit.
